(19) United States
(12) Patent Application Publication
(10) Pub. No.: US 2022/0401017 A1
(43) Pub. Date: Dec. 22, 2022

(54) POSTURE DETECTION DEVICE AND NECKLACE

(71) Applicant: HANGZHOU ZHIXING TECHNOLOGY CO., LTD., HANGZHOU (CN)

(72) Inventors: ZIYU LIU, HANGZHOU (CN); TAO BAI, DALIAN (CN); KAIWEN DENG, TANGSHAN (CN)

(21) Appl. No.: 17/355,102

(22) Filed: Jun. 22, 2021

(51) Int. Cl. A61B 5/00; G06F 1/16

(52) U.S. Cl. CPC A61B 5/4561; G06F 1/163

(57) ABSTRACT

The present disclosure provides a posture detection device and a necklace, which relates to a field of automatic control, the posture detection device is worn on a body of a user, and the posture detection device includes a sensing device, a processor, and a reminding device. The sensing device is connected to the processor, and the sensing device obtains information of back posture changes of the body of the user and sends the information to the processor. The processor is connected to the reminding device, the processor processes the information, generates a reminding signal, and sends the reminding signal to the reminding device, the reminding device starts a vibration mode according to the reminding signal.

### TECHNICAL FIELD

The present disclosure relates to a field of automatic control, in particular to a posture detection device and a necklace.

### BACKGROUND

In a mobile Internet era, popularity of electronic terminals has brought many health problems to people, for example, there are more and more people working in front of computers, and many people become phubbers. People who do not have good habits are used to bow their heads when using mobile phones due to poor self-awareness, a long-term habit of bowing their heads may cause hunchback and myopia. Especially for teenagers who are in a growth and development period and need to read and write for a long time, which may have a huge impact on the teenagers' future life and work.

For problems such as hunchback and myopia, in related arts, elastic materials are added to clothes, which strong force is used to correct postures, but it is very troublesome to wear and brings discomfort to human bodies. Meanwhile, it also has great limitations in application scenarios. In addition, through video image monitoring, image recognition is used to remind users whether they have posture problems that damage their health, at present, image monitoring is relatively high in cost and may not identify accurately.

Regarding problems of high cost and low accuracy rate of the users' posture information monitoring in related arts, no effective solutions have been proposed at present.

### SUMMARY

The present disclosure provides a posture detection device and a necklace, which solves the problems raised in above-mentioned background art.

In order to achieve above at least one objective, the present disclosure provides the following technical solutions:

The posture detection device is worn on a body of a user, the posture detection device includes a sensing device, a processor, a switching device and a reminding device.

The sensing device is connected to the processor, and the sensing device obtains information of back posture changes of the body of the user and sends the information to the processor.

The processor is connected to the reminding device, and the processor processes the information of the back posture changes of the body of the user, generates a reminding signal, and sends the reminding signal to the reminding device, the reminding device starts one of reminding modes according to the reminding signal.

The switching device is connected to the processor, after the reminding device starts the one of the reminding modes according to the reminding signal, the switching device obtains a switching signal and sends the switching signal to the processor.

A circuit suspends detecting relevant angle signals by pulling a body of the posture detection device, so that the posture detection device temporarily turns off alerts to stay in a silent state; or the circuit is turned off or a delay time from detection to alert triggering is switched into, so that a switch of the reminding modes is realized.

The processor instructs the reminding device to turn off the one of the reminding modes according to the switching signal.

Optional, the posture detection device further includes a charging battery and a wireless charging coil.

The charging battery is disposed in the body of the posture detection device, and the charging battery is connected to both the processor and the wireless charging coil.

The wireless charging coil is disposed in the body of the posture detection device, and the wireless charging coil is configured to charge the charging battery.

Optional, the posture detection device further includes a display device, the display device is connected to the processor, and the display device presents at least one working state of the posture detection device.

Optional, the posture detection device further includes a wireless communication device, the wireless communication device is connected to the processor, and the wireless communication device is configured to provide communication and connection between the posture detection device with at least one peripheral device.

A necklace, the necklace includes a hanging rope and a necklace body, the necklace body is connected to the hanging rope, and a posture detection device is disposed in a cavity of the necklace body, the posture detection device includes a sensing device, a processor, and a reminding device. The sensing device is connected to the processor, and the sensing device obtains information of back posture changes of a body of a user and sends the information to the processor.

The processor is connected to the reminding device, the processor processes the information of the back posture changes of the body of the user, generates a reminding signal, and sends the reminding signal to the reminding device, the reminding device starts one of reminding modes according to the reminding signal.

Optional, the posture detection device includes a switching device, after the reminding device starts the one of the reminding modes according to the reminding signal, the switching device obtains a switching signal and sends the switching signal to the processor.

The processor instructs the reminding device to turn off the one of the reminding modes according to the switching signal.

Optional, the posture detection device further includes a charging battery and a wireless charging coil.

The charging battery is disposed in a body of the posture detection device, and the charging battery is connected to both the processor and the wireless charging coil.

The wireless charging coil is disposed in the body of the posture detection device, and the wireless charging coil is configured to charge the charging battery.

Optional, the posture detection device further includes a display device.

The display device is connected to the processor, and the display device presents at least one working state of the posture detection device.

Optional, the posture detection device further includes a wireless communication device.

A circuit suspends detecting relevant angle signals by pulling a body of the posture detection device, so that the posture detection device temporarily turns off alerts to stay in a silent state; or the circuit is turned off or a delay time from detection to alert triggering is switched into, so that a switch of the reminding modes is realized, the wireless communication device is connected to the processor, and the wireless communication device is configured to provide communication and connection between the posture detection device with at least one peripheral device.

The present disclosure has following advantages: the present disclosure provides the posture detection device and the necklace, the posture detection device is worn on the body of the user, and the posture detection device includes the sensing device, the processor, and the reminding device. The sensing device is connected to the processor, and the sensing device obtains the information of the back posture changes of the body of the user and sends the information to the processor. The processor is connected to the reminding device, the processor processes the information of the back posture changes of the body of the user, generates the reminding signal, and sends the reminding signal to the reminding device, the reminding device starts the one of the reminding modes according to the reminding signal, which solves the problems of high cost and low accuracy rate of the users' posture information monitoring, reduces detection cost of user's posture information, improves accuracy rate, and promptly reminds users to correct body postures.

### BRIEF DESCRIPTION OF DRAWINGS

FIG. 1 is a first structural block diagram of a posture detection device according to one embodiment of the present disclosure.

FIG. 2 is a schematic diagram of direction coordinates of a body of a user according to one embodiment of the present disclosure.

FIG. 3 is a second structural block diagram of the posture detection device according to one embodiment of the present disclosure.

FIG. 4 is a third structural block diagram of the posture detection device according to one embodiment of the present disclosure.

FIG. 5 is a fourth structural block diagram of the posture detection device according to one embodiment of the present disclosure.

FIG. 6 is a schematic diagram of a necklace according to one embodiment of the present disclosure.

FIG. 7 is a schematic diagram of a circuit principle of the posture detection device according to one embodiment of the present disclosure.

FIG. 8 is a first flow chart of a method for detecting body postures according to one embodiment of the present disclosure.

FIG. 9 is a second flow chart of a method for detecting body postures according to one embodiment of the present disclosure.

FIG. 10 is a third flow chart of a method for detecting body postures according to one embodiment of the present disclosure.

FIG. 11 is a flowchart of a sleep method of the posture detection device according to one embodiment of the present disclosure.

FIG. 12 is a flowchart of a step counting method of the posture detection device according to an embodiment of the present disclosure.

### DETAILED DESCRIPTION

Embodiments of the present disclosure are described in details below, it need to be understood that the embodiments described below are used to illustrate and explain the present disclosure and are not intended to limit the present disclosure.

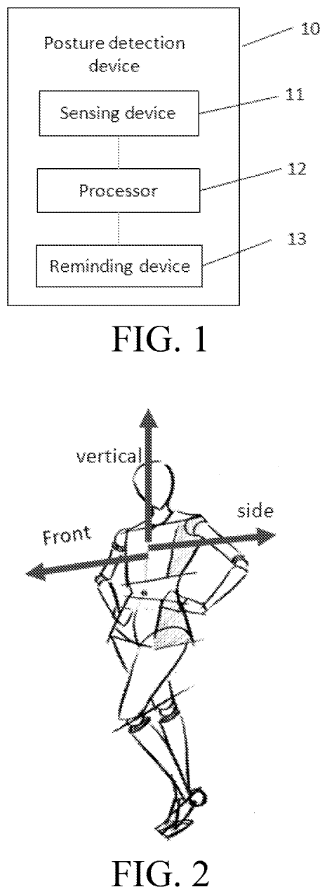

A posture detection device, as shown in FIG. 1, the posture detection device 10 is worn on a body of a user, the posture detection device 10 may be a wearable article such as a backpack, a necklace, clothes, and glasses, etc., and the posture detection device 10 includes: a sensing device 11, a processor 12, and the reminding device 13.

The sensing device 11 obtains first posture information and sends the first posture information to the processor 12, and the first posture information presents back posture changes of the body of the user. The sensing device 11 may be a three-axis sensor or a six-axis sensor, etc. For example, the three-axis sensor may include three gyroscopes and three accelerometers.

FIG. 2 is a schematic diagram of direction coordinates of a body of a user according to one embodiment of the present disclosure, as shown in FIG. 2, directions of the body of the user include a front direction, a vertical direction, and a side direction. Bending of user's body back postures in each direction may be detected by the sensing device 11.

The processor 12 compares the first posture information with a preset standard posture information, when the first posture information is greater than the standard posture information, the processor 12 generates a reminding signal and sends the reminding signal to the reminding device 13. When the sensing device 11 is a three-axis acceleration sensor, the three-axis acceleration sensor monitors that an angular motion change exceeds a preset change value on a plane formed by the front direction and the vertical direction of the body of the user, which determines that a user's back has a bending action. For example, the sensing device 11 detects that an angle of the angular motion change is twenty degrees, the preset change value is eighteen degrees, and the twenty degrees is greater than the eighteen degrees, which determines that the user has a hunchback. The processor 12 generates a reminding signal and sends the reminding signal to the reminding device 13. It needs to be noted that the sensing device 11 may also obtain the angular motion changes of other directions of the body of the user to respectively determine whether the user has a bending action in three directions.

The reminding device 13 starts one of reminding modes according to the reminding signal. It needs to be noted that the one of the reminding modes is only a way to remind the user, but may also use vibrations from a vibration motor, sounds from a speaker, light flashing and other ways to remind the user. When the one of the reminding modes is a vibration mode, the posture detection device 10 generates a vibrating sensation which is sensed by the body of the user, so as to remind that a wearer is in a hunchback state. The reminding device 13 may be a vibration feedback device such as a linear vibration motor. It needs to be noted that after the posture detection device 10 sends out a vibration alarm, the user changes a posture by adjusting the posture, the sensing device 11 obtains real-time angular motion information, and when posture information of angular motion information is less than standard posture information, the reminding device 13 stops the vibrations, the user may also stop vibration reminding by controlling the switching device on the posture detection device 10.

Through the above-mentioned posture detection device 10 worn on the body of the user, the sensing device obtains posture change information of a back of the body of the user, and compares the posture change information with the preset standard posture information, when the posture change information is greater than the standard posture information, that is, when a bending posture of a back posture of the body of the user is greater than a certain angle, the reminding device 13 reminds that the user has a hunchback or lowers a head by means of vibration, etc., which solves the problems of high cost and low accuracy rate of the user's posture information monitoring, reduces detection cost of user's posture information, improves accuracy rate, and promptly reminds users to correct body postures.

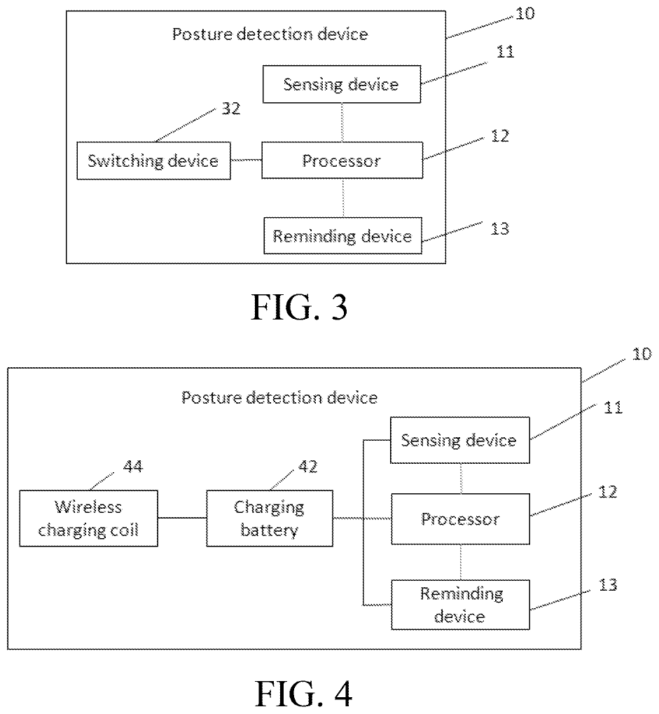

In one embodiment, FIG. 3 is a second structural block diagram of the posture detection device according to one embodiment of the present disclosure, as shown in FIG. 3, the posture detection device 10 further includes a switching device 32.

After the reminding device 13 starts the one of the reminding modes according to the reminding signal, the switching device 32 obtains a switching signal and sends the switching signal to the processor 12. The processor 12 instructs the reminding device 13 to turn off the one of the reminding modes according to the switching signal. The switching device 32 may be a physical button or a touch key, so that the user can conveniently turn off the one of the reminding modes in time, for example, when the one of the reminding modes is a vibration mode, the switching device 32 is the touch key, and after the vibration mode is started, the user touches the touch key to turn off the vibration mode.

In one embodiment, FIG. 4 is a third structural block diagram of the posture detection device according to one embodiment of the present disclosure, as shown in FIG. 4, the posture detection device 10 further includes a charging battery 42 and a wireless charging coil 44.

The charging battery 42 is disposed in a body of the posture detection device 10, and the charging battery 42 is respectively connected to the processor 12 and the wireless charging coil 44. The wireless charging coil 44 is disposed in the body of the posture detection device 10, and the wireless charging coil 44 is configured to charge the charging battery 42. In some specific application scenarios, the posture detection device 10 is a waterproof device, and may not have an external charging interface, and may only be charged through wireless charging, which improves the waterproofness of the posture detection device 10. The posture detection device 10 may also be provided with an external charging interface and a wireless charging method, which is convenient for the user to choose more charging methods. In one embodiment, the posture detection device 10 may be powered by a battery as a power source.

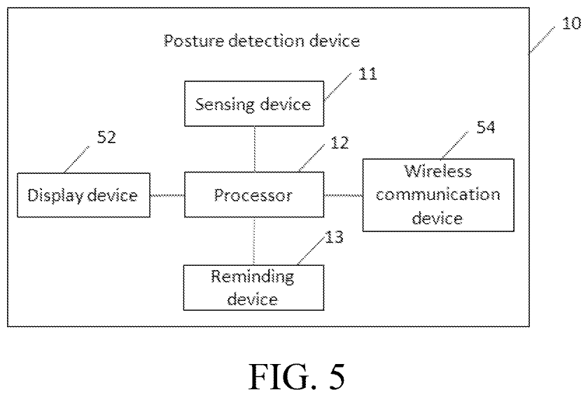

In one embodiment, FIG. 5 is a fourth structural block diagram of the posture detection device according to one embodiment of the present disclosure, as shown in FIG. 5, the posture detection device 10 further includes a display device 52, for example, the display device 52 may be a liquid crystal display, which presents power or detected data, etc. of the posture detection device 10. The posture detection device 10 further includes a wireless communication device 54, the wireless communication device 54 is connected to the processor 12, and the wireless communication device 54 is configured to provide communication and connection between the posture detection device with at least one peripheral device. For example, a mobile phone terminal installs an application corresponding to the posture detection device 10, and the posture detection device 10 communicates with the mobile terminal through the wireless communication device 54, the application performs data transmission of setting information and detection information with the posture detection device 10. In addition, communication modes of the posture detection device 10 are not limited to a wireless communication mode. After the posture detection device 10 is provided with a corresponding communication hardware module, the communication modes may also be a Wi-Fi communication or a mobile communication network, etc., which is convenient for the user to control and statistics posture data of the posture detection device 10.

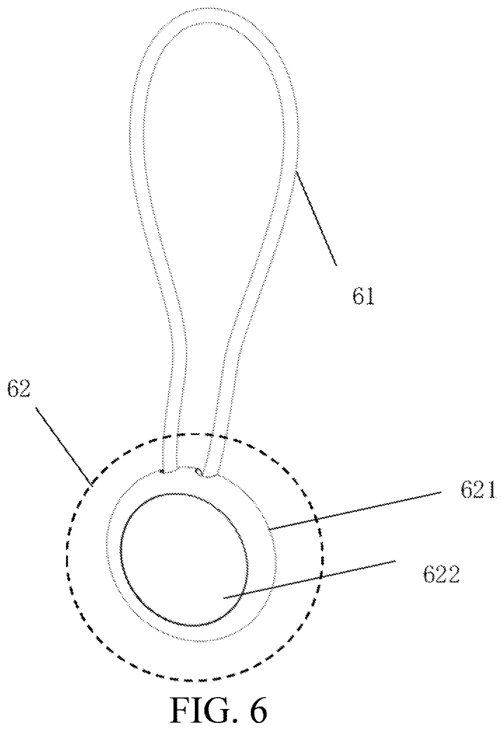

In an actual application embodiment, the posture detection device 10 may be a necklace-shaped entity, as shown in FIG. 6, a necklace includes a hanging rope and a necklace body, a posture detection device 10 is disposed in a cavity of the necklace body 62 (a pendant of the necklace), the posture detection device 10 includes a sensing device 11, a processor 12, and a reminding device 13. The necklace is hung on a neck of a user through the hanging rope 61, and the necklace body 62 is in close contact with a human body.

The sensing device 11 obtains first posture information and sends the first posture information to the processor 12, and the first posture information presents back posture changes of the body of the user.

The processor 12 compares the first posture information with a preset standard posture information, when the first posture information is greater than the standard posture information, the processor 12 generates a reminding signal and sends the reminding signal to the reminding device 13.

The reminding device 13 starts one of reminding modes according to the reminding signal.

In addition, in one embodiment, as shown in FIG. 6, the necklace body 62 includes a housing 621 of the necklace and a control region 622 of the necklace. The switching device 32 is disposed on the control region 622. The switching device 32 may be a physical button or a touch key, so that the user can conveniently turn off the one of the reminding modes in time, for example, when the one of the reminding modes is a vibration mode, and the control region 622 is a touch sensing region of the touch key, and after the vibration mode is started, the user touches the control region 622, the processor 12 turns off the vibration mode.

The necklace is worn on the neck of the user while the necklace works, which is used in a natural way. A shape of the housing of the necklace does not affect circuit work, and the necklace as a worn article may be decoratively designed to enhance its functionality. The posture detection device 10 records correct human motion states and posture change information, and achieves real-time recording and reminding through real-time monitoring. A hunchback reminding function is a main function of the posture detection device 10, and vibration feedback is natural and silent, and does not disturb surrounding people.

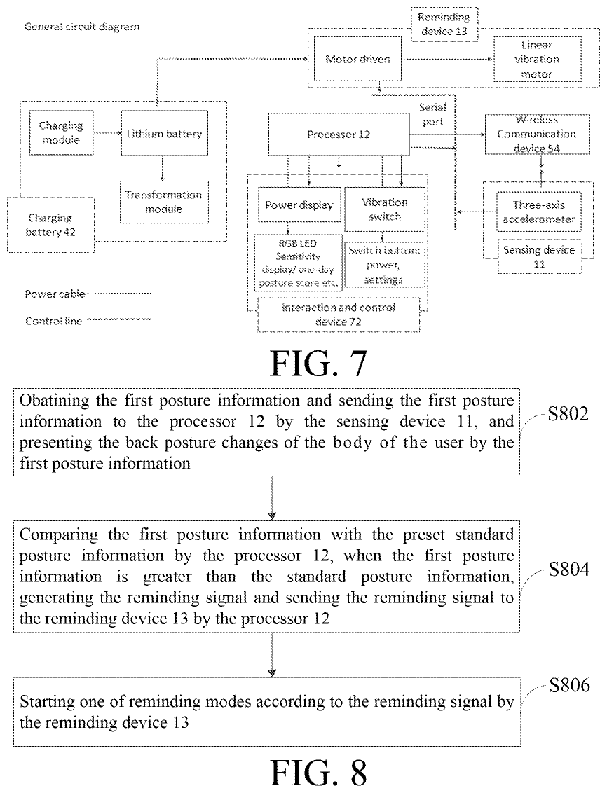

In one embodiment, FIG. 7 is a schematic diagram of a circuit principle of the posture detection device according to one embodiment of the present disclosure, the posture detection device 10 includes: a sensing device 11, a processor 12, a reminding device 13, a charging battery 42, a wireless communication device 54 and an interaction and control device 72. The charging battery 42 supplies power for each device through a power line. The processor 12 controls the reminding device 13, the interaction and control device 72, the wireless communication device 54 and the sensing device 11 through a control line. The Interaction and control device 72 can activate a button switch at a low power or a set process, or turn off the button switch. The interaction and control device 72 presents amount of power, sensitivity of posture information, or a one-day posture score, etc., which are displayed by means of red, green and blue light-emitting diodes (RGB-LED).

In one embodiment, the present disclosure provides a body posture detection method, which is applied to the posture detection device 10, the posture detection device 10 is worn on the body of the user, and the posture detection device 10 includes: a sensing device 11, a processor 12 and a reminding device 13.

A circuit suspends detecting relevant angle signals by pulling the body of the posture detection device, so that the posture detection device temporarily turns off alerts to stay in a silent state; or the circuit is turned off or a delay time from detection to alert triggering is switched into, so that a switch of the reminding modes (sensitivity) is realized.

FIG. 8 is a first flow chart of a method for detecting body postures according to one embodiment of the present disclosure, as shown in FIG. 8, the method includes following steps:

Step S802, obtaining the first posture information and sending the first posture information to the processor 12 by the sensing device 11, and presenting the back posture changes of the body of the user by the first posture information.

Step S804, comparing the first posture information with the preset standard posture information by the processor 12, when the first posture information is greater than the standard posture information, generating the reminding signal and sending the reminding signal to the reminding device 13 by the processor 12.

Step S806, starting the one of the reminding modes according to the reminding signal by the reminding device 13.

Through the above-mentioned method for detecting body postures, posture change information of a back of the body of the user is obtained, and the posture change information is compared with the preset standard posture information, when the posture change information is greater than the standard posture information, that is, when a bending posture of a back posture of the body of the user is greater than a certain angle, the user is reminded that the user has the hunchback or lowers the head by means of vibrations, etc., which solves the problems of high cost and low accuracy rate of the user's posture information monitoring, reduces detection cost of user's posture information, improves accuracy rate, and promptly reminds users to correct body postures.

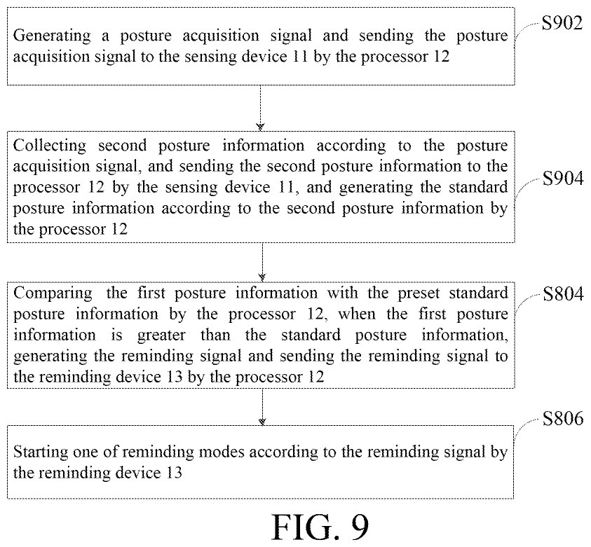

In one embodiment, FIG. 9 is a second flow chart of a method for detecting body postures according to one embodiment of the present disclosure, as shown in FIG. 9, the method includes following steps:

Step S902, generating a posture acquisition signal and sending the posture acquisition signal to the sensing device 11 by the processor 12.

Step S904, collecting second posture information according to the posture acquisition signal, and sending the second posture information to the processor 12 by the sensing device 11, and generating the standard posture information according to the second posture information by the processor 12.

Through steps S902 to S904, the user needs to set the standard posture information when initializing the posture detection device 10 during an initial use or during a use process. For example, during the initial use, the user is reminded through displaying or vibrations that current is an acquisition time of the standard posture information, so that the user sits upright, and the second posture information obtained by the sensing device 11 at this time is the standard posture information. Furthermore, postures of different users are customized to adapt to the different users, which improves the accuracy rate of detection.

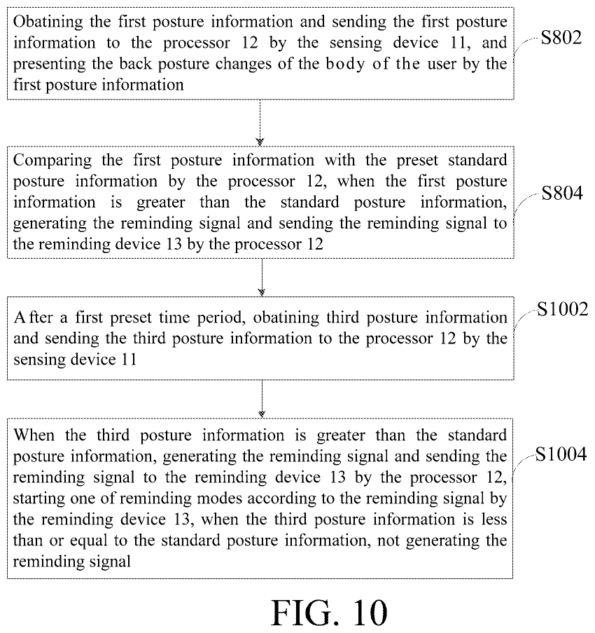

In one embodiment, FIG. 10 is a third flow chart of a method for detecting body postures according to one embodiment of the present disclosure, as shown in FIG. 10, the method includes following steps:

Step S1002: after a first preset time period, obtaining third posture information and sending the third posture information to the processor 12 by the sensing device 11.

Step S1004: when the third posture information is greater than the standard posture information, generating the reminding signal and sending the reminding signal to the reminding device 13 by the processor 12, starting the one of the reminding modes according to the reminding signal by the reminding device 13, when the third posture information is less than or equal to the standard posture information, not generating the reminding signal.

Through steps S1002 to S1004, the user may have some special application scenarios when the user uses the posture detection device 10, for example, the user bends over to pick up something. Therefore, it is necessary to adjust trigger sensitivity of the posture detection device 10. In normal work, after the reminding signal is triggered for the first preset time period, the third posture information of the user is obtained and compared with the standard posture information, a situation that the user's posture information temporarily and greatly changes in special scenarios is eliminated, and the reminding device 13 is prevented from being triggered by mistakes to start the one of the reminding modes.

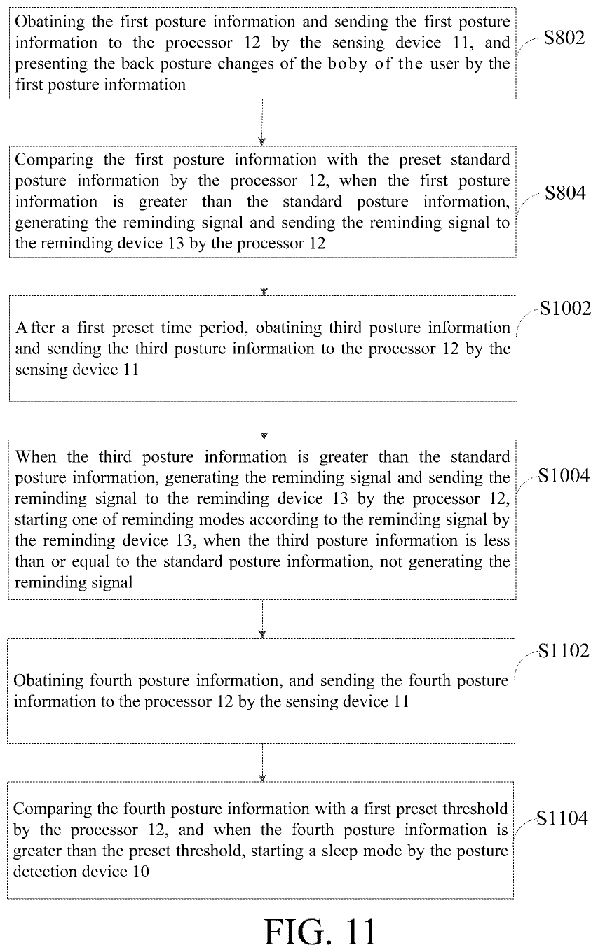

In one embodiment, FIG. 11 is a flowchart of a sleep method of the posture detection device according to one embodiment of the present disclosure, as shown in FIG. 11, the method includes following steps:

Step S1102, obtaining fourth posture information, and sending the fourth posture information to the processor 12 by the sensing device 11.

Step S1104, comparing the fourth posture information with a first preset threshold by the processor 12, and when the fourth posture information is greater than the preset threshold, starting a sleep mode by the posture detection device 10.

Through steps S1002 to S1004, when the user uses the posture detection device 10, the posture detection device 10 may be removed and not used for a period of time, in order to prolong use time of the posture detection device 10 and save power, a situation when the posture detection device 10 is horizontally laid may be set, for example, when the first preset threshold is eighty degrees and the fourth posture information angle change is eighty-nine degrees, the posture detection device 10 is determined to be horizontally laid, and the posture detection device 10 starts the sleep mode, or after a horizontal state lasts for a period of time, the posture detection device 10 restarts the sleep mode, and the sleep mode is prevented from being triggered by mistakes.

In addition, in one embodiment, the posture detection device 10 only monitors spatial angle changes within a certain range when monitoring the hunchback, and the posture detection device 10 stops current monitoring work after the spatial angle changes are exceeded, excluding hunchback reminding when the spatial angle changes are close to ninety degrees or zero degrees, which also prolongs the use time of the posture detection device 10 and saves power.

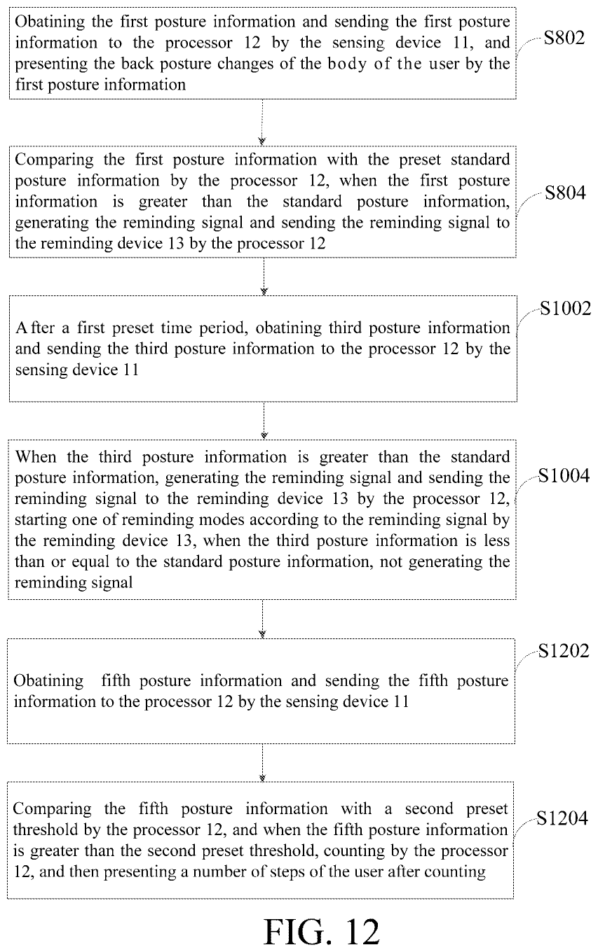

In one embodiment, FIG. 12 is a flowchart of a step counting method of the posture detection device according to an embodiment of the present disclosure, as shown in FIG. 12, the method includes following steps:

Step S1202, obtaining fifth posture information and sending the fifth posture information to the processor 12 by the sensing device 11.

Step S1204, comparing the fifth posture information with a second preset threshold by the processor 12, and when the fifth posture information is greater than the second preset threshold, counting by the processor 12, and then presenting a number of steps of the user after counting.

The posture detection device 10 realizes a step counting function. When the sensing device 11 is the three-axis acceleration sensor, when the three-axis acceleration sensor monitors that the posture detection device 10 has a forward acceleration in the front direction corresponding to a human body and center of gravity changes up and down twice in the vertical direction and presents a certain periodic change, the certain periodic change is determined as an effective step in walking state, and the step counting function is realized by counting acceleration change peak values corresponding to the vertical direction of a human body. The step counting data is recorded and executed by the processor 12 to realize whole step counting algorithm. A debugging mechanism is set up in the step counting algorithm to ensure effectiveness of step counting. The debugging mechanism includes: one of the conditions for determining whether the user walks is to walk continuously instead of a single moving position, so changes of the center of gravity corresponding to the vertical direction of the human body are continuous and reach a certain number of cycles. Because fluctuation of walking is larger than fluctuation of activities such as riding in a car, a second condition is that the acceleration change peak values during process are higher than a particular value. At the same time, the fluctuation of walking is smaller than fluctuation of running, so a third condition is that the acceleration change peak values are also less than an another particular value. Because a limit of human walking speed and a limit of nerve reflection, a fourth condition is that intervals of the acceleration change peak values is to be within a certain time range. Step counting is used as an auxiliary function, and the posture detection device 10 may be used as a data collection terminal and sends statistical data to a connected mobile phone.

A person who skilled in the art would understand that all or part of processes in the above-mentioned method embodiments can be implemented by instructing relevant hardware through a computer program. The computer program is stored in a non-volatile computer readable storage medium, when the computer program is executed, it may include the processes of the above-mentioned method embodiments. Any reference to memory, storage, database, or other media used in the embodiments provided in the present disclosure may include non-volatile and/or volatile memory. Non-volatile memory may include read only memory (ROM), programmable ROM (PROM), electrically programmable ROM (EPROM), electrically erasable programmable ROM (EEPROM), or flash memory. Volatile memory may include random access memory (RAM) or external cache memory. As an illustration and not a limitation, RAM is available in many forms, such as static RAM (SRAM), dynamic RAM (DRAM), synchronous DRAM (SDRAM), double data rate SDRAM (DDRSDRAM), enhanced SDRAM (ESDRAM), synchronous chain Channel (Synchlink) DRAM (SLDRAM), memory bus (Rambus) direct RAM (RDRAM), direct memory bus dynamic RAM (DRDRAM), and memory bus dynamic RAM (RDRAM), etc.

Finally, it needs to be noted that above descriptions are only preferred embodiments of the present disclosure and are not intended to limit the present disclosure. Although the present disclosure has been described in detail with reference to above embodiments, those of ordinary skill in the art should understand that they can still modify the technical solutions described in the foregoing embodiments, or equivalently replace some of the technical features. Any modification, equivalent replacement, improvement, etc. made within spirit and principle of the present disclosure should be included in a protection scope of the present disclosure.

What is claimed is:

1. A posture detection device, worn on a body of a user, comprising:
a sensing device;
a processor;
a switching device; and
a reminding device;
wherein the sensing device is connected to the processor, and the sensing device obtains information of back posture changes of the body of the user and sends the information to the processor;
the processor is connected to the reminding device, and the processor processes the information of the back posture changes of the body of the user, generates a reminding signal, and sends the reminding signal to the reminding device; the reminding device starts one of reminding modes according to the reminding signal;
the switching device is connected to the processor; after the reminding device starts the one of the reminding modes according to the reminding signal, the switching device obtains a switching signal and sends the switching signal to the processor; a circuit suspends detecting relevant angle signals by pulling a body of a posture detection device, so that the posture detection device temporarily turns off alerts to stay in a silent state; or the circuit is turned off or a delay time from detection to alert triggering is switched into, so that a switch of the reminding modes is realized; and
the processor instructs the reminding device to turn off the one of the reminding modes according to the switching signal.

2. The posture detection device according to claim 1, further comprising a charging battery and a wireless charging coil; wherein the charging battery is disposed in the body of the posture detection device, and the charging battery is connected to both the processor and the wireless charging coil; and the wireless charging coil is disposed in the body of the posture detection device, and the wireless charging coil is configured to charge the charging battery.

3. The posture detection device according to claim 1, further comprising a display device, wherein the display device is connected to the processor, and the display device presents at least one working state of the posture detection device.

4. The posture detection device according to claim 1, further comprising a wireless communication device; wherein the wireless communication device is connected to the processor, and the wireless communication device is configured to provide communication and connection between the posture detection device with at least one peripheral device.

5. A necklace, comprising:
a hanging rope; and
a necklace body;
wherein the necklace body is connected to the hanging rope, and a posture detection device is disposed in a cavity of the necklace body, the posture detection device comprises a sensing device, a processor, and a reminding device; the sensing device is connected to the processor, and the sensing device obtains information of back posture changes of a body of a user and sends the information to the processor; the processor is connected to the reminding device, the processor processes the information of the back posture changes of the body of the user, generates a reminding signal, and sends the reminding signal to the reminding device, the reminding device starts one of reminding modes according to the reminding signal.

6. The necklace according to claim 5, wherein the posture detection device further comprises a switching device, after the reminding device starts the one of the reminding modes according to the reminding signal, the switching device obtains a switching signal and sends the switching signal to the processor; the processor instructs the reminding device to turn off the one of the reminding modes according to the switching signal.

7. The necklace according to claim 5, wherein the posture detection device further comprises a charging battery and a wireless charging coil; the charging battery is disposed in a body of the posture detection device, and the charging battery is connected to both the processor and the wireless charging coil; the wireless charging coil is disposed in the body of the posture detection device, and the wireless charging coil is configured to charge the charging battery.

8. The necklace according to claim 5, wherein the posture detection device further comprises a display device; the display device is connected to the processor, and the display device presents at least one working state of the posture detection device.

9. The necklace according to claim 5, wherein the posture detection device further comprises a wireless communication device; a circuit suspends detecting relevant angle signals by pulling a body of the posture detection device, so that the posture detection device temporarily turns off alerts to stay in a silent state; or the circuit is turned off or a delay time from detection to alert triggering is switched into, so that a switch of the reminding modes is realized, the wireless communication device is connected to the processor, and the wireless communication device is configured to provide communication and connection between the posture detection device with at least one peripheral device.
